(19) United States
(12) Patent Application Publication
(10) Pub. No.: US 2009/0004175 A1
(43) Pub. Date: Jan. 1, 2009

(54) Methods for Optimizing Forming Vlla-Based Hemostatic Treatment

(75) Inventor: Lars Hansen, Frederiksberg C (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordick Health Care A/G, Zurich (CH)

(21) Appl. No.: 11/632,607

(22) PCT Filed: Jul. 14, 2005
(86) PCT No.: PCT/EP05/53390

§ 371 Date: Feb. 19, 2008

Related U.S. Application Data
(60) Provisional application No. 60/589,503, filed on Jul. 20, 2004.

(30) Foreign Application Priority Data
Jul. 16, 2004 (DK) PA 2004 01117

(51) Int. Cl. A61K 38/48; C12Q 1/68

(52) U.S. Cl. 424/94.64; 435/6

(57) ABSTRACT

The present invention relates to pharmacogenomic methods for optimizing prevention and treatment of bleeding episodes using therapeutic proteins such as, e.g., Vitamin K-dependent clotting factors.

### FIELD OF THE INVENTION

The present invention relates to pharmacogenomic methods for optimizing prevention and treatment of bleeding episodes using therapeutic proteins such as, e.g., Vitamin K-dependent clotting factors.

### BACKGROUND OF THE INVENTION

Haemostasis is a complex physiological process that ultimately results in the arrest of bleeding. This is dependent on the proper functioning of three main components: blood vessels (especially the endothelial lining), coagulation factors, and platelets. Once a haemostatic plug is formed, timely activation of the fibrinolytic system is equally important to prevent further unnecessary haemostatic activation. Any malfunction of this system may lead to clinical bleeding such as, e.g., haemorrhagic diatheses of varying severity.

In most physiological situations, haemostasis is triggered by exposure to the circulation of tissue factor (TF) at sites of injury, followed in succession by (i) binding of plasma Factor VII (FVII) to TF and its proteolytic conversion to activated Factor VII (FVIIa); (ii) binding of Factor X to the TF-FVIIa complex and its proteolytic conversion to activated Factor X (FXa); (iii) proteolytic conversion by FXa of prothrombin to thrombin; and (iv) the generation of a complex between tissue factor pathway inhibitor (TFPI) and FXa, followed by binding of the TFPI:FXa complex to TF-FVIIa, which attenuates FXa activation of thrombin and limits the flux of thrombin generated via the TF pathway. The relatively small amount of thrombin produced during this phase results in the activation of FXI to FXIa (which activates Factor IX to FIXa) and the activation of Factor V on the surface of platelets and the further activation of Factor X. These events further promote the formation of sufficient amounts of thrombin (the so-called “thrombin burst”) to convert fibrinogen into fibrin, thereby stabilizing an initial platelet plug and resulting in appropriate haemostasis.

Several of the vitamin K-dependent clotting factors are currently used therapeutically. For example, activated recombinant human factor VII (rFVIIa) is currently indicated for the treatment of bleeding episodes in haemophilia A or B subjects with inhibitors to Factor VIII or Factor IX. When given in sufficiently high (pharmacological) doses, rFVIIa can bind independently of TF to activated platelets and initiate local thrombin generation which is important for the formation of the initial haemostatic plug.

Factor VIIa also finds use in controlling many other types of bleeding, such as, e.g., bleeding occurring in non-haemophiliac patients as a result of trauma, surgical procedures, or other pathological conditions.

The increased use of Factor VIIa as a therapeutic modality raises the possibility that individuals having different genotypes may respond differently to Factor VIIa treatment.

Thus, there is a need in the art for improved methods and compositions that allow optimization of Vitamin K-dependent clotting factor treatment by taking into account a patient's genetic makeup.

### SUMMARY OF THE INVENTION

The present invention provides methods for selecting a therapeutic regimen for treatment of bleeding episodes in a human, which are carried out by in vitro analyzing a biological sample obtained from the individual for the presence of a predetermined genetic polymorphic pattern, wherein the polymorphic pattern comprises a polymorphism in one or more genes and wherein the presence of the pattern correlates with an outcome of a therapeutic regimen for the treatment. In some embodiments, the therapeutic regimen involves administration of a clotting factor. In some embodiments, the clotting factor is a vitamin K-dependent clotting factor, such as, e.g., is Factor VIIa or a Factor VIIa equivalent.

In some embodiments, the polymorphic pattern comprises a polymorphism in one or more genes encoding proteins involved in blood coagulation or fibrinolysis, including, without limitation, Tissue Factor (TF), Factor VII, Factor IX, Factor X, TF pathway inhibitor (TFPI), prothrombin, Factor XI, thrombomodulin, Factor VIII, Factor XIII, plasminogen activator, plasminogen, protein C, Factor V, platelet glycoprotein Ib, platelet fibrinogen receptor glycoprotein IIb-IIIa; plasminogen activator inhibitor-1 (PAI-1); Factor VI; protein C inhibitor; protein S inhibitor; tissue plasminogen activator (tPA) inhibitor; and protease nexin II (PNII). In some embodiments, the proteins are one or more of: Factor V, Factor VII, and prothrombin. In some embodiments, the polymorphic pattern comprises one or more of: Factor V R506Q, Prothrombin g2021a, and Factor VII-323 del/ins.

In some embodiments, the presence of the polymorphic pattern correlates with an increased sensitivity to exogenously administered FVIIa. In some embodiments, the presence of said polymorphic pattern correlates with a decreased sensitivity to exogenously administered FVIIa.

In some embodiments, the analyzing step comprises amplifying nucleic acid isolated from said biological sample and detecting said polymorphic pattern in said amplified nucleic acid.

In some embodiments, at least one of the polymorphisms is present in both alleles of the gene in the patient.

In some embodiments, the therapeutic regimen comprises one or more of: (i) the quantity of FVIIa or a FVIIa equivalent administered to the subject; (ii) the timing of administration of FVIIa or a Factor VIIa equivalent and (iii) the use of non-FVIIa therapeutic modalities.

In some embodiments, the analyzing step may involve one or more of: treatment with a restriction enzyme that differentiates between allelic variants; single-stranded conformation polymorphism; allele specific nucleic acid hybridization; primer specific extension; oligonucleotide ligation assay; and DNA microarrays.

In another aspect, the invention provides kits for screening individuals who are candidates for Factor VIIa-mediated treatment of bleeding episodes, which comprise: an oligonucleotide primer that amplifies the region encompassing a polymorphism in a gene to produce an amplification product, wherein the polymorphism comprises all or part of a polymorphic pattern and wherein the presence of the polymorphic pattern has been correlated with an outcome of a predetermined regimen for Factor VIIa-mediated treatment. The kits may further comprise a detectable marker for the amplification product. The kits may also comprise a plurality of oligonucleotide primers, wherein each primer amplifies a region encoding a polymorphism in a different gene and wherein the genes encode proteins involved in blood coagulation or fibrinolysis. In some embodiments, the proteins are one or more of: Tissue Factor (TF), Factor VII, Factor IX, Factor X, TF pathway inhibitor (TFPI), prothrombin, Factor XI, thrombomodulin, Factor VIII, Factor XIII, plasminogen activator, plasminogen, protein C, Factor V, platelet glycoprotein Ib, platelet fibrinogen receptor glycoprotein IIb-IIIa; plasminogen activator inhibitor-1 (PAI-1); Factor VI; protein C inhibitor; protein S Inhibitor; tissue plasminogen activator (tPA) inhibitor; and protease nexin II (PNII). In some embodiments, the polymorphic pattern comprises one or more of: Factor V R506Q, Prothrombin g2021a, and Factor VII-323 del/ins.

In other aspects, the invention provides methods for identifying a subject as a candidate for a particular therapeutic regimen to treat a bleeding episode; methods for determining if a patient will benefit from a particular therapeutic regimen to treat a bleeding episode; methods for predicting the pharmacokinetics of Factor VIIa; methods for predicting outcome of Factor VIIa treatment; methods for inferring a response to Factor VIIa treatment; methods for treating bleedings; methods for identifying polymorphic patterns that predict outcomes of Factor VIIa treatment; and methods for identifying individuals as suitable for a clinical trial involving Factor VIIa treatment.

In other aspects, the invention provides methods for promoting sales of haemostatic agents (including, without limitation, Factor VIIa and Factor VIIa equivalents), which are carried out by screening individuals to detect one or more predetermined genetic polymorphic patterns, wherein the polymorphic pattern comprises a polymorphism in one or more genes and wherein the presence of the pattern correlates with an outcome of a therapeutic regimen for the treatment.

In other aspects, the invention relates to a pharmaceutical product comprising: (a) a clotting factor in a container; and (b) a notice associated with said container in a form prescribed by a governmental agency regulating the manufacture, use, or sale of pharmaceuticals, which note is reflective of the approval of said agency that said clotting factor can or should be used in a treatment regime which depends on the presence or absence of one or more predetermined genetic polymorphic pattern, wherein the polymorphic pattern comprises a polymorphism in one or more genes and wherein the presence of the pattern correlates with an outcome of a therapeutic regimen for the treatment.

### DETAILED DESCRIPTION OF THE INVENTION

The present invention provides methods and kits for optimizing the treatment of bleeding episodes using therapeutic proteins, such as, e.g., vitamin K-dependent factors. The methods are carried out by analyzing the genotype of patients for the presence of particular genetic variations, including polymorphisms that, either singly or in combination, correlate with specific treatment outcomes using particular treatment regimens that involve therapeutic administration of one or more vitamin K-dependent clotting factors. In other aspects, the invention provides methods for identifying a subject as a candidate for a particular therapeutic regimen to treat a bleeding episode, methods for determining if a patient will benefit from a particular therapeutic regimen to treat a bleeding episode, methods for predicting the pharmacokinetics of the clotting factor, methods for predicting the outcome of treatment, and methods for inferring a response to the treatment. Predictive polymorphic patterns:

In practicing the invention, a biological sample is obtained from a patient and analyzed for the presence of specific polymorphic sequences within one or more genes. The polymorphism may represent a single nucleotide polymorphism (SNP), i.e., a change of a single nucleotide relative to a reference sequence, or may represent insertions, deletions, or substitutions of more than a single residue. The polymorphism may be located anywhere in the genome, including, e.g., within a protein-coding region, an intron, or in a 5′ or 3′ regulatory region of a particular gene. The polymorphism may or may not result in an alteration of the sequence of the primary translation product and/or the final protein product of the gene; and/or, the polymorphism may or may not result in some alteration of the level of expression and/or of the biological activity of the protein product of the gene.

The reference sequence, also referred to as the wild-type sequence or wild-type allele, refers to the most common sequence in a particular population. An allelic variant, therefore, refers to alternative forms of a gene that may contain a polymorphism useful in the present invention.

Alleles occupy the same locus or position on homologous chromosomes. When a subject has two identical alleles of a gene, the subject is said to be homozygous for allele of that gene; conversely, when a subject has two different alleles of a gene, the subject is said to be heterozygous for the allele of that gene. Alleles of a specific gene can differ from each other in one or more nucleotides, or by insertion or deletion of nucleotides. As used herein, the term “allelic variant” is used interchangeably with “polymorphism”.

Also encompassed by the present invention are polymorphisms within a chromosomal region that has not been assigned to a particular gene, as well as longer-range heritable alterations in chromosome structure. Furthermore, relevant polymorphisms may be those that are in genes that are not apparently physiologically related to coagulation or fibrinolysis, but wherein the polymorphism is located so that it is physically/genetically linked to a particular phenotype with respect to haemostatic treatment.

As used herein, a polymorphic pattern refers to the presence or absence of one or more polymorphisms in an individual. The invention encompasses any polymorphic pattern that can be used to predict how an individual patient will respond to a particular treatment regimen.

In one set of embodiments, the polymorphic pattern comprises polymorphism(s) in one or more genes that are involved in blood coagulation and/or fibrinolysis. Non-limiting examples of such genes include: Tissue Factor (TF), Factor VII, Factor VII activating protein (FSAP), Factor IX, Factor X, TF pathway inhibitor (TFPI), prothrombin, Factor XI, thrombomodulin, Thrombospondin (TSP-1), Factor VIII, Factor XIII, plasminogen activator, fibrinogen, plasminogen, protein C, Factor V, fibrinogen, platelet glycoprotein Ib, platelet fibrinogen receptor glycoprotein IIb-IIIa (GPIIIa); plasminogen activator inhibitor-1 (PAI-1); Factor VI; protein C; protein C inhibitor; protein S; protein S inhibitor; protein Z, tissue plasminogen activator (tPA) inhibitor; protease nexin II (PNII); and interleukin 1 receptor antagonist (IL1RN).

In some embodiments, the polymorphic pattern comprises polymorphism(s) in one or more genes that are implicated in cardiovascular function and/or cardiovascular disease. Non-limiting examples of such genes include: angiotensin I-converting enzyme, Cholesterol Ester Transfer Protein (CETP), and Apolipoprotein ε4.

In some embodiments, the polymorphic pattern comprises polymorphism(s) in one or more of: Factor V, Factor VII, and prothrombin. In some embodiments, the polymorphic pattern comprises one or more of the following polymorphisms: Factor V R506Q (also known as Factor V g1691a or Factor V Leiden) (Bertina et al., Nature 369:64, 1994), Factor V Hong Kong (g1090a); Factor V Cambridge (g1091c) Factor V g1628a; Factor V R2 allele (a4070gA) (Dogulu et al., Thromb. Res. 111:389, 2003).; Prothrombin g2021a (Poort et al., Blood 88:3698, 1996); prothrombin Gla29Gly (E29G); prothrombin T165M; prothrombin polymorphisms in introns 4, 5, 7, 13; Factor VII 323 del/ins (Iacoviello et al., N. Engl. J. Med. 338:79, 1998; Girelli et al., N. Engl. J. Med. 343: 774, 2000); Factor VII R353Q; Factor VII A2 (10 bp-insertion in 5′ region); Factor VII L204P; as well as Factor VII polymorphisms in the promoter, intron 4 and intron 7.

In some embodiments, the polymorphic pattern comprises polymorphism(s) in one or more of, without limitation: FSAP, such as, e.g., the Marburg I or Marburg II variants (U.S. Pat. No. 5,994,080); tPA, such as, e.g., the Alu-h I allele (U.S. Pat. No. 5,994,080); GPIIIa, such as, e.g., Leu33 or Pro33 polymorphism (U.S. Pat. No. 5,955,266); IL1RN (U.S. patent application 2003/0187335); Factor XIII V34L; Protein Z G79A; CETP CETP) TaqIB (B1B1 gentoype); Platelet gpia C807T; promoter insertions such as, e.g., Tissue Factor promoter 1208 insertion; b-fibrinogen 455 g/a; tissue plasminogen activator (tPA) −7351 T/T (homozygous genotype); plasminogen activator inhibitor-1 (PAI-1)-promoter 4G/5G polymorphism; and von Willebrand factor (VWF) promoter (−1793c/g); methylene tetrahydrofolate reductase (MTHFR) C677T; Thrombomodulin A455V; ATP-Binding Cassette Transporter (ABCA1) R219K; Angiotensin-Converting Enzyme (ACE) 287 bp deletion of Alu sequence in intron 16 (D allele); and P selecin T715P.

The invention encompasses any polymorphic pattern that correlates with an increased or decreased sensitivity to a particular haemostatic treatment and/or that is predictive of outcome of particular treatment regimens. It will be understood that relevant polymorphisms may be identified using any means. For example, a candidate polymorphism (i.e., one that could form part of a polymorphic pattern that can be used predictively in planning treatment regimens) may be identified by its appearance in a family of patients whose members exhibit a characteristic defect in some aspect of coagulation or fibrinolysis. Alternatively, a candidate polymorphism may be identified by large-scale DNA sequencing of nucleic acid derived from patients who have exhibited a particular outcome to a treatment regimen. Statistical analysis is then used to support or negate correlations between particular polymorphisms or combinations of polymorphisms and treatment outcomes (see below.)

### Methods for Detecting Polymorphic Patterns

In practicing the present invention, any method may be used that detects a relevant polymorphism, and any combination of methods may be used, as appropriate, to establish the presence or absence of particular polymorphisms that together comprise polymorphic patterns. Non-limiting examples of nucleic acid-based detection methods include: restriction enzyme site analysis; single-stranded conformation polymorphism analysis; allele-specific hybridization; primer-specific extension; direct nucleic acid sequencing; oligonucleotide ligation assays, as well as the use of oligonucleotide or DNA-based microarrays.

Typically, a biological sample is obtained from the patient and nucleic acid sequences encompassing a particular polymorphism are amplified by Polymerase Chain Reaction (PCR) using the nucleic acid as a template and appropriate oligonucleotide primers. The PCR products can be subjected to any useful method to detect a polymorphism. In one embodiment, the PCR products are subjected to an electrophoretic assay (such as gel electrophoresis or capillary electrophoresis) to determine the relative size of the PCR product. In another embodiment, the PCR products are probed with a nucleic acid sequence (fluorescently-labelled or labelled in any other detectable manner) that is specific for a particular polymorphism (e.g., an allele-specific probe). In another embodiment, the PCR products are sequenced using techniques known in the art, including commercially available sequencing kits, to determine if a polymorphism is present in the sample. Other sequencing technologies such as Denaturing High Pressure Liquid Chromatography or: mass spectroscopy may also be employed.

In other embodiments, detection of a polymorphism can be performed by using differential restriction enzyme digestion or by using Single Stranded Conformation Polymorphism (SSCP) techniques. Primer-specific extension may also be used as a detection method. It will be understood that different methods may be usefully employed in different cases, depending at least in part upon the nature of the difference between the polymorphic sequence and the reference sequence.

In one series of embodiments, the nucleic acid may comprise mRNA isolated from a biological sample, or DNA transcribed from the mRNA using, e.g., RT-PCR, that is labelled (usually via fluorescent or radioactive label) and used to hybridize to a multiplicity of sequences such as those displayed on a microarray chip; depending on the nature of the sequences in the microarray, a variety of polymorphisms may be detectable using a single biological sample from the patient.

The invention also encompasses the use of detection methods that do not involve direct analysis of nucleic acid sequences. In some instances, the existence of particular genotypes may be reflected in another measurable physiological parameter, such as, e.g., the level of a particular protein in the circulation or in some other body fluid or tissue; a functionality related to, e.g., coagulation or fibrinolysis, or any other property that is detectable using a method that can be applied to a patient in need of treatment for a bleeding disorder. In some cases, such as, e.g., when a particular polymorphism or polymorphic pattern is known to exist within a family, a predetermined clinical parameter known to be reflective of the polymorphism may be used to assess the presence or absence of the polymorphic pattern in a given individual of the family.

Combinations of any of the above methods or other useful detection methods may also be used.

### Treatment Regimens and Outcomes

As used herein, a treatment regimen refers to administration of one or active agents to a patient who is experiencing a bleeding episode or is anticipated may suffer from a bleeding episode and/or from any ancillary consequences of a bleeding episode. In one series of embodiments, the active agent is one or more of a clotting factor, including, without limitation, clotting factors such as FVIIa. The invention may be applied to treatment of any type of bleeding episode or for steps taken to achieve any other therapeutic benefit associated with prevention or treatment of a bleeding episode.

Typically, the active agent(s) comprise FVIIa, such as, e.g., human Factor VIIa, as disclosed, e.g., in U.S. Pat. No. 4,784,950 (wild-type Factor VII). The term “Factor VIIa” refers to Factor VII polypeptides have been proteolytically processed (typically, between residues 152 and 153) to yield their respective bioactive forms, as well as to Factor VIIa equivalents that may be biologically active in an uncleaved form.

Factor VIIa equivalents include, without limitation, Factor VII polypeptides that have either been chemically modified relative to human Factor VIIa and/or contain one or more amino acid sequence alterations relative to human Factor VIIa. Such equivalents may exhibit different properties relative to human Factor VIIa, including stability, phospholipid binding, altered specific activity, and the like.

In one series of embodiments, a Factor VIa equivalent includes polypeptides that exhibit at least about 10%, preferably at least about 30%, more preferably at least about 50%, and most preferably at least about 70%, of the specific biological activity of human Factor VIIa. For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of a Factor VIIa equivalent to produce Factor Xa in a system comprising TF embedded in a lipid membrane and Factor X. (Persson et al., J. Biol. Chem. 272:19919-19924, 1997); (ii) measuring Factor X hydrolysis in an aqueous system; (iii) measuring the physical binding of Factor VIIa or a Factor VIIa equivalent to TF using an instrument based on surface plasmon resonance (Persson, FEBS Letts. 413:359-363, 1997) and (iv) measuring hydrolysis of a synthetic substrate by Factor VIIa and/or a Factor VIIa equivalent.

Examples of factor VII equivalents include, without limitation, wild-type Factor VII, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A -FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, and K316Q/L305V/V158T/E296V/M298Q/K337A-FVII.

Bleeding refers to extravasation of blood from any component of the circulatory system. A bleeding episode encompasses unwanted, uncontrolled and often excessive bleeding in connection with surgery, trauma, or other forms of tissue damage, as well as unwanted bleedings in subjects having bleeding disorders. Bleeding episodes may occur in subjects having a basically normal coagulation system but experiencing a (temporary) coagulophathy, as well as in subjects having congenital or acquired coagulation or bleeding disorders. In subjects having a defective platelet function, the bleedings may be analogous to bleedings caused by haemophilia in that the haemostatic system, as in haemophilia, lacks or has abnormal essential clotting “compounds” (e.g., platelets or von Willebrand factor protein). In subjects who experience extensive tissue damage, such as, for example, in association with surgery or trauma, the normal haemostatic mechanism may be overwhelmed by the demands of immediate haemostasis and they may develop excessive bleeding in spite of a basically (pre-trauma or pre-surgery) normal haemostatic mechanism. Such subjects, who further often are multi-transfused, may develop a transient coagulopathy as a result of the bleeding and/or transfusions (i.e., a dilution of coagulation proteins, increased fibrinolysis, and lowered number of platelets due to the bleeding and/or transfusions). Bleeding may occur in organs such as the brain, inner ear region, and eyes, presenting limited possibilities for surgical intervention and thus potential difficulty in achieving satisfactory haemostasis. Similar problems may arise in the process of taking biopsies from various organs (liver, lung, tumour tissue, gastrointestinal tract) as well as in laparoscopic surgery and radical retropubic prostatectomy. Common to all these situations is the difficulty in providing haemostasis by surgical techniques (sutures, clips, etc.) which also is the case when bleeding is diffuse (e.g., haemorrhagic gastritis and profuse uterine bleeding). Bleeding may also occur in subjects being treated with anticoagulants, in whom a defective haemostasis has been induced by the therapy and in whom bleedings are often acute and profuse. Anticoagulant therapy is often given to prevent thromboembolic disease. Such therapy may include heparin, other forms of proteoglycans, warfarin or other forms of vitamin K-antagonists, inhibitors of coagulation proteins, as well as aspirin and other platelet aggregation inhibitors, such as, e.g., antibodies or other inhibitors of GP IIb/IIIa activity. The bleeding may also be due to so-called thrombolytic therapy, which may comprise combined treatment with an antiplatelet agent (e.g., acetylsalicylic acid), an anticoagulant (e.g., heparin), and a fibrinolytic agent (e.g., tissue plasminogen activator, tPA). Bleeding episodes also include, without limitation, uncontrolled and excessive bleeding in connection with surgery or trauma in subjects having acute haemarthroses (bleedings in joints), chronic haemophilic arthropathy, haematomas, (e.g., muscular, retroperitoneal, sublingual and retropharyngeal), bleedings in other tissue, haematuria (bleeding from the renal tract), cerebral haemorrhage, surgery (e.g., hepatectomy), dental extraction, and gastrointestinal bleedings (e.g., UGI bleeds). Bleeding episodes may be associated with inhibitors against factor VIII; haemophilia A; haemophilia A with inhibitors; haemophilia B; deficiency of factor VII; deficiency of factor XI; thrombocytopenia; deficiency of von Willebrand factor (von Willebrand's disease); severe tissue damage; severe trauma; surgery; laparoscopic surgery; acidosis; haemodilution; consumption coagulopathies; hyperfibrinolysis; hyopthermia; haemorrhagic gastritis; taking biopsies; anticoagulant therapy; upper gastroentestinal bleedings (UGI); or stem cell transplantation.

In the context of the present invention, treatment encompasses both prevention of bleeding (including, without limitation, prevention of an expected bleeding, such as, for example, as might occur during or consequent to a surgical procedure), as well as regulation of an already occurring bleeding, such as, for example, bleeding that results from trauma, with the purpose of inhibiting or minimizing the bleeding. The bleeding may be at an identified site or may be at an undetermined site. Prophylactic administration of a preparation comprising a vitamin K-dependent clotting factor, such as, e.g., FVIIa or a FVIIa-related polypeptide, is thus included in treatment.

Treatment regimens for treating bleeding episodes, such as, e.g., mediated by Factor VIIa, may comprise one or more of the following variables, without limitatoin: dosage amount, number of doses, timing of administration, mode of administration, and combination with other bioactive compounds or other therapeutic modalities. Treatment regimens also encompass diagnostic procedures and assessment of clinical indicators that may be performed prior to the first administration of, e.g., FVIIa or at any time during or after a course of such administration.

Factor VIIa or the Factor VIIa equivalent may be administered to a patient as a single dose or in a staged series of doses which together comprise an effective amount for preventing or treating bleeding. An effective amount of Factor VIIa or the Factor VIIa equivalent refers to the amount of Factor VIIa or equivalent which, when administered in a single dose or in the aggregate of multiple doses, or as part of any other type of defined treatment regimen, produces a measurable statistical improvement in outcome, as evidenced by at least one relevant clinical parameter. When Factor VIIa equivalents are administered, an effective amount may be determined by comparing the coagulant activity of the Factor VIIa equivalent with that of Factor VIIa and adjusting the amount to be administered proportionately to the predetermined effective dose of Factor VIIa.

Non-limiting examples of treatment regimens include administration of Factor VIa or a Factor VIIa equivalent to prevent bleeding within about 24 hours prior to surgery, such as, e.g., within about 8 hours, within about 2 hours, or within about 1 hour; administration of Factor VITa or a Factor VIIa equivalent to treat bleeding within about 6 hours after the start of bleeding, such as, e.g., within about 4 hours, within about 2 hours, or within about 1 hour.

Administration of a single dose refers to administration of an entire dose of Factor VIa or the Factor VIIa equivalent as a bolus over a period of less than about 5 minutes. In some regimens, the administration occurs over a period of less than about 2.5 minutes, and, in some, over less than about 1 min.

In some regimens, following administration of a single dose of Factor VIIa or a Factor VIIa equivalent according to the invention, the patient receives no further Factor VIa or Factor VIIa equivalent for an interval of at least about 30 minutes. In some regimens, the post-administration interval is at least about 45 minutes, such as at least about 1 hour, at least about 1.5 hours, or at least about 2 hours.

In one regimen, for example: (i) the patient receives a first amount of Factor VIIa or Factor VIIa equivalent comprising at least about 40 ug/kg; (ii) after a period of at least about 30 minutes, a second amount of Factor VIIa or Factor VIIa equivalent is administered, the amount comprising at least about 40 ug/kg; and (iii) after a period of at least about 30 minutes from administration of the second dose, a third amount of Factor VIIa or Factor VIIa equivalent is administered, the amount comprising at least about 40 ug/kg. After a period of at least about 30 minutes following the administration of the third amount, the patient may then receive a further (fourth) amount of Factor VIIa or Factor VIIa equivalent comprising at least about 40 ug/kg.

In other regimens, the first amount of Factor VIIa or Factor VIIa equivalent comprises at least about 100 ug/kg, such as at least about 150 ug/kg or at least about 200 ug/kg; in other regimens, the second amount of Factor VIIa or Factor VIIa equivalent comprises at least about 75 ug/kg, such as at least about 90 ug/kg; in other regimens, the third (and optionally fourth) amount of Factor VIIa or Factor VIIa equivalent comprises at least about 75 ug/kg, such as at least about 90 ug/kg.

In one regimen, the first dose comprises about 200 ug/kg, the second dose about 100 ug/kg, and the third (and optionally fourth) dose about 100 ug/kg.

In other regimens, the patient receives the second amount of Factor VIIa or Factor VIIa equivalent after a period of at least about 45 minutes from the first administration, such as at least about 1 hour, at least about 1.5 hours, at least about 2 hours, at least about 2.5 hours, or at least about 3 hours.

In other regimens, the patient receives the third (and optionally fourth) amount of Factor VIIa or Factor VIIa equivalent after a period of at least about 45 minutes from the previous administration, such as at least about 1 hour, at least about 1.5 hours, at least about 2 hours, at least about 2.5 hours, or at least about 3 hours.

In one regimen, for example, the patient receives a first dose comprising about 200 ug/kg; after a period of about 1 hour, the patient receives a second dose comprising about 100 ug/kg, and after a period of about 3 hours from the first dose, the patient receives a third dose comprising about 100 ug/kg.

Treatment regimens may also encompass combination treatments, such as, e.g., the combined use of Factor VIIa and/or a Factor VIIa equivalent with other agents that promote coagulation, including, without limitation, one or more of: Factor XIII (see, e.g., WO 01/85198); inhibitors of tissue factor pathway inhibitor (TFPI inhibitors) (see, e.g., WO 01/85199); Factor IX (see, e.g., WO 02/062376); thrombin activatable fibrinolysis inhibitor (TAFI) (see, e.g., PCT/DK02/00734; PAI-1 (see, e.g., PCT/DK02/00735; Factor V (see, e.g., PCT/DK02/00736); protein C inhibitors (see, e.g., PCT/DK02/00737); thrombomodulin (see, e.g., PCT/DK02/00738); protein S inhibitors (see, e.g., PCT/DK02/00739); tissue plasminogen activator inhibitors (see, e.g., PCT/DK02/00740); α2-antiplasmin (see, e.g., PCT/DK02/00741); aprotinin (see, e.g., PCT/DK02/00742); tranexamic acid (see, e.g., PCT/DK02/00751); ε-aminocaproic acid (see, e.g., PCT/DK02/00752); prothrombin, thrombin, FX, and fibrinogen.

Factor VIIa-mediated treatment regimens may also encompass assessment of the patient's haemostatic status, which, in turn, may be reflected in one or more clinical parameters, including, e.g., relative levels of one or more circulating coagulation factors; amount of blood lost; rate of bleeding; hematocrit; ex vivo clot-forming ability; and the like. In this context, a treatment regimen may comprise (i) administering a first dose of Factor VIIa or a Factor VIIa equivalent; (ii) assessing the patient's coagulation status after a predetermined time; and (iii) based on the assessment, administering a further dose of Factor VIIa or Factor VIIIa equivalent if necessary. Steps (ii) and (iii) may be repeated until satisfactory haemostasis is achieved.

Treatment regimens also include the route of administration of Factor VIIa or a Factor VIIa equivalent, including, without limitation, administration by intravenous, intramuscular, subcutaneous, mucosal, and pulmonary routes of administration.

Outcomes of Factor VIIa-mediated treatment may be measured and expressed in any conventional manner. Non-limiting examples of outcome parameters include: the amount of blood or blood products that a patient needs to receive to resolve a bleeding episode; the time (measured either from the start of bleeding or from initiation of treatment) until the bleeding episode is resolved; and the occurrence and severity of complications that result at least in part from multiple blood transfusions, including, e.g., Pulmonary embolism (PE), Acute Respiratory Distress Syndrome (ARDS), Disseminated Intravascular Coagulation (DIC), Acute Myocardial Infarction (AMI), Cerebral Thrombosis (CT), Systemic Inflammatory Response Syndrome (SIRS), infections, Multiple Organ Failure (MOF), and Acute Lung Injury (ALI), including death caused by one or more of these syndromes.

The present invention encompasses the correlation with particular polymorphic patterns of any clinical indicator that is useful in assessing and/or designing treatment regimens. For example, the correlation of a patient's polymorphic pattern with likelihood that the patient will develop antibodies to a therapeutically administered clotting factor can be useful in designing a treatment regimen for the patient.

### Identifying Relevant Polymorphic Patterns and Correlations

The present invention encompasses establishing associations between a patient's genotype (i.e., the presence or absence of particular polymorphic patterns) and the effect of a treatment regimen that involves administration of one or more vitamin K-dependent clotting factors. It will be understood that such associations may be established using conventional methods, such as, e.g., by performing genetic analyses on groups of patients who have undergone such treatment using different treatment regimens, and collecting data on: (a) genotypes; (b) treatment regimens; (c) outcomes, and (d) any other relevant information (such as, e.g., demographic, family history, and the like). Statistical analyses may then be performed using conventional methods, such as, e.g., the statistical system SAS (SAS Institute, Inc., Gary N.C.). Statistical models for the dependence of treatment outcome on treatment regimen, genotype information, and other relevant information may be applied that enable the predictive value of polymorphisms or polymorphic patterns to be assessed. In particular, associations between genotype and particular treatments may be modelled by including appropriate treatment by genotype interaction terms in such models. The analysis may involve the use of model selection procedures to identify relevant polymorphic patterns.

### Kits

The present invention encompasses kits that can be used to screen individuals who are candidates for treatment of bleeding episodes using a vitamin K-dependent clotting factor, such as, e.g., Factor VIIa or Factor VIIa equivalents. The kits can be used to determine the presence or absence in a patient of polymorphic patterns, which information can then be used in making clinical decisions as to the suitability of a Factor VIIa-mediated treatment regimen.

The reagents in the kit can include oligonucleotide primers to the appropriate regions of one or more genes in order to amplify nucleic acids from a biological sample using PCR, such as, e.g., primers that amplify the region encompassing a polymorphism that itself comprises part of a polymorphic pattern relevant for the present invention. The kit may further include nucleic acid probes useful in determining the presence of a polymorphism in the gene(s). The kit may also include electrophoretic markers such as a 200 bp ladder. Other components of the kit can include nucleotides, enzymes and buffers that can be used in a detection method. In one embodiment, a kit of the invention may include primers for amplifying the region surrounding one or more of: the nucleotides encoding residue 506 of the Factor V gene; the nucleotides encoding residue 323 of the Factor VII gene; and/or nucleotide 2021 of the prothrombin gene. In another series of embodiments, the kit will include any diagnostic reagents used to detect the level of a relevant protein or marker. including, without limitation, immunological reagents or reagents for performing enzymatic assays. The kit will also include detailed instructions for carrying out the method for detecting the presence of the relevant polymorphism. The following are intended as non-limiting examples of the present invention:

### EXAMPLE 1

### Methods for Identifying Polymorphic Patterns Relevant to Factor VIIa-Mediated Treatments

The following methods are used to identify individuals carrying one or more genetic polymorphisms whose presence or absence may be used by a clinician in determining an appropriate Factor VIIa-mediated treatment.

a. Factor V R506Q

The following primers are used for the amplification of three exons of the FV gene. Forward (F) and reverse (R) primers and amplification conditions are: exon 7, F7 5′-TGTCTTTCTGTCCTAAC-3′, R7 5′-TCTTGAACCTTTGCCCA-3′ (annealing at 42° C.×35 cycles); exon 10, F10 5′-TGCCCAGTGCTTAACAAGACCA-3′, R10 5′-TGTTATCACACTGGTGCTAA-3′ (annealing at 55° C.×36 cycles); exon 13, F13 5′-CAAGTCCTTCCCCACAGATATA-3′, R13 5′-AGATCTGCAAAGAGGGGCAT-3′ (annealing at 57° C.×30 cycles) (using a GeneAmp PCR System 9700 thermocycler (Applied Biosystems, Foster City, Cslif.). The amplification reaction is performed in a 50-^{μ}l volume that contained 300 ng of genomic DNA, 0.2 mM of each primer, 0.2 mM dNTPs, 2.5 mM MgCl₂, 2U/100 ^{μ}l of thermostable DNA polymerase (Amplitaq 5U/^{μ}l, Invitrogen, Carlsbad, Calif.). The specificity and the size of amplified fragments are checked by 4% polyacrylamide gel electrophoresis (PAGE). After PCR amplification, the 1090A→G or 1091G→C polymorphism in exon 7 and 1628G→A and 1691G→A polymorphisms in exon 10 are determined by direct sequence analysis. The genotype of R2 allele in exon 13 are detected by restriction fragment length polymorphism using the RsaI restriction enzyme (RFLP).

b Prothrombin g2021a

The prothrombin g2021 allele is detected using any of the methods described in, e.g., Angelini et al., J. Thrombosis and Thrombolysis 16:189, 2003; or Poort et al., Blood, 10:3698, 1996.

c. Factor VII-323 del/ins

The Factor VII-323 del/ins allele is detected using, e.g., the method described in Girelli et al., N. Engl. J. Med. 343:774, 2000.

### EXAMPLE 2

Identifying Polymorphic Patterns that Predict the Outcome of Factor VIIa-Mediated Treatment Regimens

The following study is performed to identify polymorphic patterns for use in the present invention:

A randomized double-blind parallel-group placebo-controlled clinical trial evaluating the effect of Factor VIIa in the prevention of bleeding in patients undergoing a particular type of surgery is used. One outcome that is measured is the requirement for perioperative transfusions. Biological samples taken from the patients are used to screen for polymorphisms and/or polymorphic patterns; the information obtained is incorporated into conventional statistical models (using, e.g, the SAS system) to identify associations between particular polymorphisms or polymorphic patterns and one or more clinical indicators. Details of the treatment regimen are also factored into the analyses.

### Example 3

### Effect of Thrombophilic Gene Variants on Factor VII-Mediated Coagulation Parameters

The following study was performed to analyze the effect of of three thrombophilic gene variants on Factor VIIa coagulation activity in vivo. A single centre, randomised, double blind, cross-over trial was performed in which 40 healthy subjects were administered Factor VIIa.

### Methods:

1. Effect of FVIIa administration on coagulation parameters: Subjects were administered 90 ug/kg body weight of Factor VIIa as a slow intravenous injection of 2 minutes duration. Blood samples were taken prior to injection and at 30, 60, and 720 minutes after injection, and were analyzed for the following: platelet count (measured as G/L); concentration of fibrinogen (expressed as g/L, Sta Compact, Stago); aPTT (prothrombin time, expressed in seconds, Sta Compact Stago); concentration of fibrin dimers (“D-dimers”, expressed as ng/ml, Mini-Vidas, BioMerieux, Durham N.C., htto://www.biomerieux-usa.com/company/index.htm); and concentration of antithrombin (expressed as %, Sta Compact, Stago).

2. Genotyping: A simple finger stick blood sample was obtained and blood droplets were applied to a SAFEspot blood collection card and allowed to dry. DNA was extracted and genotyped for the following mutations: g1691a Factor V (Leiden); TT677 methylene tetrahydofolate reductase (MTFHR); and g2021a prothrombin.

### Results:

Thrombophilic gene variants. The prevalence of Factor V Leiden and Factor II g20210a were 4/45 (9%), and MTHFR C677T: 22/45 (49%). Excluding screening failures the prevalence in randomised subjects were: Factor V Leiden and Factor II g20210a: 3/40 (8%), and MTHFR C677T: 18/40 (45%). The genotypes for Factor II and Factor V were combined due to the low number of carriers, all of whom were heterozygotes. A subject was considered a carrier of combined genotype if he was a carrier of any of the genotypes

### Effect of Genotype on Coagulation Parameters.

The analysis was made as an ANOVA including a fixed period and treatment effect and a random subject effect to account for the two measurements made on each subject. For the coagulation data the baseline value of the variable was included as an additional factor.

The results of the analyses are summarised below. Least squares (LS) means are presented for the genotypes. These means correspond to the mean estimate adjusted for all factors in the model, based on an equal distribution of all factors. Therefore the value or level is more theoretical than directly derived from the actual data. The values are thus best used to compare different levels of the genotypes, since these differences correspond to the expected difference.

Factor II+V—D-Dimers at 60 minutes, p-value=0.004
Estimated LS means for WT/MUT=263.16
Estimated LS mean for WT/WT=171.16
   Factor II+V—D-Dimers at 720 minutes, p-value=0.004
Estimated LS mean for WT/MUT=595.0
Estimated LS mean for WT/WT=301.5
   MTHFR—Fibrinogen at 720 minutes, p-value=0.04
Estimated LS mean for MUT/MUT=2.84
Estimated LS mean for WT/MUT=2.42
Estimated LS mean for WT/WT=2.60
   MTHFR—PT at 720 minutes, p-value=0.06
Estimated LS mean for MUT/MUT=10.39
Estimated LS mean for WT/MUT=10.82
Estimated LS mean for WT/WT=10.78

Comparison of pre-and post-treatment parameters indicated that there was a statistically significant treatment effect of the pooled Factor II+Factor V mutant genotypes on D-Dimers at 60 (p=0.03) and 720 minutes (p<0.001) after administration of Factor VIIa. For Fibrinogen and PT at 720 minutes there was a statistically significant treatment effect of the MTHFR C677T genotype (p<0.001).

The three subjects carrying thrombophilic gene variants Factor V Leiden (one subject) and Factor II G20210A (two subjects) responded with significant higher plasma concentrations of D-dimers after exposure to Factor VIIa. Importantly, the two carriers of the Factor II G20210A polymorphism were the only two subjects who presented with high values of D-dimers (>1000 ng/ml) during both treatment episodes, and one of the subjects (11304) still had comparatively high levels (442 ng/ml, cut off for “thigh value” 494 ng/ml) nine days after exposure.

Overall this analysis demonstrates that genetic markers such as the Factor II, Factor V, and MTHFR genotypes can be valuable in explaining and predicting treatment effects for Factor VIIa. Specifically for these data a clear statistically significant differential treatment effect was demonstrated for the carriers of the combined Factor II and Factor V genotype.

All patents, patent applications, and literature references referred to herein are hereby incorporated by reference in their entirety.

Many variations of the present invention will suggest themselves to those skilled in the art in light of the above detailed description. Such obvious variations are within the full intended scope of the appended claims.

1. A method for selecting a therapeutic regimen for treatment of bleeding episodes in a human, which comprises analyzing a biological sample obtained from the individual for the presence of a predetermined genetic polymorphic pattern, wherein the polymorphic pattern comprises a polymorphism in one or more genes and the presence of the pattern correlates with an outcome of a therapeutic regimen for the treatment.

2. A method as defined in claim 1, wherein the therapeutic regimen involves administration of a clotting factor.

3. A method as defined in claim 2, wherein the clotting factor is selected from the group consisting of a vitamin K-dependent clotting factor.

4. A method as defined in claim 3, wherein the clotting factor is Factor VIIa or a Factor VIIa equivalent.

5. A method as defined in claim 1, wherein the polymorphic pattern comprises a polymorphism in one or more genes encoding proteins involved in blood coagulation or fibrinolysis.

6. A method as defined in claim 5, wherein the polymorphic pattern comprises a polymorphism in a gene encoding a protein selected from the group consisting of: Tissue Factor (TF), Factor VII, Factor IX, Factor X, TF pathway inhibitor (TFPI), prothrombin, Factor XI, thrombomodulin, Factor VIII, Factor XIII, plasminogen activator, plasminogen, protein C, Factor V, platelet glycoprotein Ib, platelet fibrinogen receptor glycoprotein IIb-IIIa; plasminogen activator inhibitor-1 (PAI-1); Factor VI; protein C inhibitor; protein S inhibitor; tissue plasminogen activator (tPA) inhibitor; protease nexin II (PNII); and combinations of any of the foregoing.

7. A method as defined in claim 6, wherein the polymorphic pattern comprises one or more of: Factor V R506Q, Prothrombin g2021a, and Factor VII-323 del/ins.

8. A method as defined in claim 1, wherein the presence of the polymorphic pattern correlates with an increased sensitivity to exogenously administered FVIIa.

9. A method as defined in claim 1, wherein the presence of the polymorphic pattern correlates with a decreased sensitivity to exogenously administered FVIIa.

10. A method as defined in claim 1, wherein at least one of the polymorphisms is present in both alleles of the gene in the patient.

11. A method as defined in claim 1, wherein the therapeutic regimen comprises one or more of: (i) the quantity of FVIIa or a FVIIa equivalent administered to the subject; (ii) the timing of administration of FVIIa or a Factor VIIa equivalent and (iii) the use of non-FVIIa therapeutic modalities.

12. A kit for screening individuals who are candidates for Factor VIIa-mediated treatment of bleeding episodes, which comprises an oligonucleotide primer that amplifies the region encompassing a polymorphism in a gene to produce an amplification product, wherein the polymorphism comprises all or part of a polymorphic pattern and wherein the presence of the polymorphic pattern is correlated with an outcome of a predetermined regimen for Factor VIIa-mediated treatment.

13. A kit as defined in claim 12, further comprising a detectable marker for the amplification product.

14. A kit as defined in claim 12, comprising a plurality of oligonucleotide primers, wherein each primer amplifies a region encoding a polymorphism in a different gene and wherein the genes encode proteins involved in blood coagulation or fibrinolysis.

15. A kit as defined in claim 14, wherein the proteins are selected from the group consisting of: Tissue Factor (TF), Factor VII, Factor IX, Factor X, TF pathway inhibitor (TFPI), prothrombin, Factor XI, thrombomodulin, Factor VIII, Factor XIII, plasminogen activator, plasminogen, protein C, Factor V, platelet glycoprotein Ib, platelet fibrinogen receptor glycoprotein IIb-IIIa; plasminogen activator inhibitor-1 (PAI-1); Factor VI; protein C inhibitor; protein S inhibitor; tissue plasminogen activator (tPA) inhibitor; protease nexin II (PNII).

16. A kit as defined in claim 15, wherein the proteins are one or more of: Factor V, Factor VII, and prothrombin.

17. A pharmaceutical product comprising: (a) a clotting factor in a container; and (b) a product information sheet, approved by a governmental agency regulating manufacture, use or sale of pharmaceuticals associated with said container, stating the clotting factor can or should be used in a treatment regime which depends on the presence or absence of one or more predetermined genetic polymorphic pattern, wherein the polymorphic pattern comprises a polymorphism in one or more genes and wherein the presence of the pattern correlates with an outcome of a therapeutic regimen for the treatment.

18. The pharmaceutical product according to claim 17, wherein the clotting factor is of a vitamin K-dependent clotting factor.

19. The pharmaceutical product according to claim 18, wherein the clotting factor is Factor VIIa or a Factor VIIa equivalent.

20. The pharmaceutical product according to claim 16, wherein the polymorphic pattern comprises a polymorphism in one or more genes encoding proteins involved in blood coagulation or fibrinolysis.

21. The pharmaceutical product according to claim 20, wherein the polymorphic pattern comprises a polymorphism in a gene encoding a protein selected from the group consisting of: Tissue Factor (TF), Factor VII, Factor IX, Factor X, TF pathway inhibitor (TFPD, prothrombin, Factor XI, thrombomodulin, Factor III, Factor XIII, plasminogen activator, plasminogen, protein C, Factor V, platelet glycoprotein Ib, platelet fibrinogen receptor glycoprotein IIb-IIIa; plasminogen activator inhibitor-1 (PAI-1); Factor VI; protein C inhibitor; protein S inhibitor; tissue plasminogen activator (tPA) inhibitor; protease nexin II (PNII); and combinations of any of the foregoing.

22. The pharmaceutical product according to claim 21, wherein the polymorphic pattern comprises one or more of: Factor V R506Q, Prothrombin g2021a, and Factor VII-323 del/ins.
